(19) United States
(12) Patent Application Publication
(10) Pub. No.: US 2007/0178113 A1
(43) Pub. Date: Aug. 2, 2007

(54) Superantigen conjugate

(76) Inventors: B. Thomas Backstrom, Wellington (NZ); Andrea Louise McNeill, Wellington (NZ)

Correspondence Address: OCCHIUTI ROHLICEK & TSAO, LLP, 10 FAWCETT STREET, CAMBRIDGE, MA, 02138, US

(21) Appl. No.: 11/604,057

(22) Filed: Nov. 22, 2006

Related U.S. Application Data
(60) Provisional application No. 60/739,573, filed on Nov. 22, 2005.

(51) Int. Cl. A61K 39/00; A61K 38/46; C12N 9/16; C12P 21/06; A61K 38/17

(52) U.S. Cl. 424/185.1; 435/69.1; 435/320.1; 435/325; 530/350; 435/196; 514/7; 514/8; 424/94.6

(57) ABSTRACT

This invention relates to treatment of multiple sclerosis using an immunomodulatory construct including a T cell binding defective superantigen coupled to one or more myelin-associated proteins, peptides, or functionally equivalent variants thereof.

### FIELD

The present invention relates to agents and methods for the treatment of multiple sclerosis.

### BACKGROUND

Multiple Sclerosis (MS) is an inflammatory disease which results in demyelination of the central nervous system (CNS). MS affects approximately one in 1,500 New Zealanders and is characterized by progressive impairment of mobility, vision, and coordination. The course of disease is variable but can be grouped into several distinct patterns: primary progressive, secondary progressive, and relapsing-remitting.

MS is considered to be an autoimmune disorder¹, which may occur due to decreased activity of natural CD4⁺/CD25⁺ regulatory T cells (Tregs)².

Discrimination between self and non-self is required to ensure correct regulation of immune responses, thus avoiding autoimmunity. The regulation of immune responses can be mediated by the naturally occurring CD4⁺/CD25⁺ regulatory T cells. In some autoimmune diseases, it has been shown that natural Tregs are necessary to prevent anti-self responses. In MS the function of Treg cells from patients may be altered².

In addition to natural CD4⁺/CD25⁺ regulatory T cells there exists one or more populations of induced regulatory T cells. While natural Tregs are generated and gain their suppressor function in the thymus, induced regulatory T cells gain theirs in the periphery. Although both natural and induced regulatory T cell populations have been shown to inhibit a number of experimental autoimmune diseases, methods to expand and activate existing antigen-specific regulatory T cells require further research.

Attempts have been made to treat experimental autoimmune encephalomyelitis (EAE) in mice and MS by administering free peptides associated with the onset or development of the disease. For example, oral, intranasal and subcutaneous delivery of encephalitogenic peptides have been shown to generate suppressor T cells, which can inhibit the onset of autoimmunity³⁻⁵. However, such treatment options have the disadvantage that large doses and/or prolonged delivery of the peptides are required for efficient suppression.

There are currently 3 types of treatments available that have been shown to change the clinical course of certain forms of MS. They are beta interferon-1b (Betaferon®), beta interferon-1a (Avonex®, Rebif®) and glatiramer acetate (Copaxone®). The interferons are naturally derived hormones involved in controlling the body's immune system whilst glatiramer acetate is a mixture of short chains of amino acids (the building blocks of proteins) which modulates the immune response in a way yet to be defined.

Current treatments such as interferons and glatiramer acetate have to be given every day (Copaxone®) to once a week (Avonex®). Furthermore, interferons treat the symptoms, not the cause of the disease, and therefore have to be used continuously.

One of the more common forms of MS is a relapsing-remitting disease, in which patients have acute episodes of disease, followed by spontaneous recovery and relapse of disease. As disease progresses, relapses become more severe, recovery becomes less complete and the residual disease worsens. The immunology behind this relapsing-remitting disease is not entirely clear. In some animal studies epitope spreading, or changes in the specificity of the activated auto-reactive cells, have been demonstrated to be associated with disease relapse^{13,14}. It has been suggested that new epitopes are present and exposed to auto-reactive T cells as a result of the tissue damage associated with disease¹⁵. In the EAE model both intra (within the same protein) and inter (between different proteins) molecular epitope spreading has been demonstrated for PLP, MBP and MOG^{15,16}. However, relapses have also been demonstrated to occur in the absence of epitope spreading in a transgenic model of EAE, in which mice express only one T cell receptor (TCR)¹⁷. In humans, epitope spreading is more difficult to demonstrate, as the analysis must be performed in the peripheral blood, where the frequency of auto-reactive T cells is low. Changes in the reactivity of T cells observed in peripheral blood could be due to the migration of auto-reactive T cells into the periphery from tissue sites, rather than the activation of T cells of a different specificity.

The potential existence of epitope spreading in autoimmune diseases causes problems in the treatment of the disease. Early treatment of disease with a therapy specific for one epitope may help prevent disease. However, if epitope spreading has already occurred, an epitope-specific treatment would not prevent relapses in disease caused by auto-reactive T cells with different specificities. Hence, when MS is diagnosed, it is likely that epitope spreading has begun. As a consequence treatments directed at one epitope would be of limited therapeutic benefit. MS may be efficiently treated by suppressing the initial disease-causing antigen and inducing bystander suppression to other auto-reactive epitopes.

Bystander suppression is the process by which administration of one antigen generates regulatory cells specific for this antigen but that suppresses disease-inducing cells specific for another antigen. Glatiramer acetate (GA, Copaxone®) is one of the therapies currently used for the treatment of MS. There is evidence that bystander suppression may occur in MS patients treated with glatiramer acetate (GA), Copaxone®. However, evidence suggests that GA may have the ability to non-specifically modulate other immune responses. While GA was initially designed to inhibit myelin basic protein (MBP)-specific responses, it has been shown to suppress the development of other autoimmune diseases including Crohn's disease and graft rejection in experimental models.

Bystander suppression has been demonstrated in an EAE model in mice. For example, mucosal administration of PLP₁₃₉₋₁₅₁ peptide is able to prevent the onset of MBP and/or intact myelin induced EAE in an IL-10 dependent manner^{19,216}. However, as noted above, treatment of EAE or MS based on the administration of free peptides has disadvantages.

Bibliographic details of the publications referred to herein are collected at the end of the description.

### OBJECT

It is an object of the present invention to provide novel agents and/or methods of treatment of multiple sclerosis, or at least to provide the public with a useful choice of either.

### STATEMENT OF INVENTION

In a first aspect of the present invention there is provided a method for the treatment of multiple sclerosis, the method comprising at the least the step of administering to a subject in need thereof an immunomodulatory construct comprising a T cell binding defective superantigen (mSag) coupled to one or more myelin-associated protein, peptide or functionally equivalent variant thereof.

Preferably, the or each myelin-associated protein, peptide or functionally equivalent variant is chosen from the group consisting of:
Myelin oligodendrocyte glycoprotein (MOG);
Myelin-associated glycoprotein (MAG);
Proteolipid protein (PLP);
Myelin basic protein (MBP);
Oligodendrocyte-specific glycoprotein (OSP);
2′,3′-Cyclic nucleotide 3′ phosphodiesterase (CNPase);
Myelin-associated oligodendrocytic basic protein (MOBP);
α-B Crystallin (αB-C);
S100β Protein;
Transaldolase-H (Tal-H);
Oligodendrocyte myelin glycoprotein (OMGP); and
Copaxone®.

Preferably, the or each myelin-associated protein, peptide, or functionally equivalent variant is chosen from the group consisting of:
Myelinoligodendrocyte glycoprotein (MOG);
Proteolipid protein (PLP);
Myelin basic protein (MBP); and
Copaxone®.

Preferably, mSag is a T cell binding defective version of a bacterial superantigen chosen from the group consisting of:
SMEZ (preferably SMEZ-2);
SPE-C; and
SEA.

Preferably, the mSag is chosen from the group consisting of:
SMEZ-2 W75L;
SMEZ-2 D42C;
SMEZ-2 W75L.D42C.K182Q;
SMEZ-2 Y 18A; and
SMEZ-2 W75L.D42C.K182Q.Y18A.

Preferably, a method of the first aspect further includes administering one or more additional agents to the subject.

In a second aspect, the present invention provides a method for the treatment of multiple sclerosis, the method comprising at the least the steps of:
collecting and purifying regulatory T cells from a patient with multiple sclerosis;
ex vivo stimulating the T cells with an immunomodulatory construct of the first statement of the invention; and
administering the stimulated T cells into the patient.

In a third aspect there is provided a method of treating multiple sclerosis induced by immune cells to a first myelin-associated protein or peptide, wherein the method includes at least the step of administering to a subject in need thereof an immunomodulatory construct comprising a T cell binding defective superantigen (mSag) coupled to one or more myelin-associated protein, peptide or functionally equivalent variant thereof which are distinct from said first myelin-associated protein or peptide.

Preferably the method is for the treatment of relapsing-remitting MS.

In a fourth aspect the invention provides an immunomodulatory construct comprising a T cell binding defective superantigen coupled to one or more myelin-associated protein, peptide or functionally equivalent variant thereof.

In a fifth aspect, there is provided a nucleic acid encoding an immunomodulatory construct of the invention.

In a sixth aspect, the invention provides a pharmaceutical composition comprising an immunomodulatory construct comprising a T cell binding defective superantigen coupled to one or more myelin-associated protein, peptide or functionally equivalent variant thereof in combination with one or more pharmaceutically acceptable carriers, adjuvants, excipients and/or diluents.

In a seventh aspect, the invention provides a kit for treatment of multiple sclerosis, the kit comprising at least an immunomodulatory construct comprising a T cell binding defective superantigen coupled to one or more myelin-associated protein, peptide or functionally equivalent variant thereof, or a pharmaceutical composition comprising same.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### FIGURES

These and other aspects of the present invention, which should be considered in all its novel aspects, will become apparent from the following description, which is given by way of example only, with reference to the accompanying figures, in which:

FIG. 1 illustrates powerful antigen-specific T cell activation by mSAg-MOG₃₅₋₅₅ and protection against the development of EAE. A: Mice were immunized to induce EAE, as described in the text, and then treated with mSag-MOG₃₅₋₅₅, mSag-OVA₃₂₃₋₃₃₉, or PBS seven days after later. Data shown are the mean clinical score from a number of experiments (for details see Table 2). B: Proliferation of MOG₃₅₋₅₅ (2D2) or OVA₃₂₃₋₃₃₉ (OT-II) specific TCR transgenic lymph node cells in response to peptide or peptide coupled to mSag.

Data shown is one of at least 4 different experiments with similar results. C: Mice were immunized to induce EAE, as in A, and then treated with varying doses of mSag-MOG₃₅₋₅₅ or MOG₃₅₋₅₅ seven days later. Data shown are the mean values from 5 mice in each group. D: Mice were immunized to induce EAE, as in A, and then treated with mSag-MOG₃₅₋₅₅ or MOG₃₅₋₅₅. Lymph node cells from protected mice (at day 40 post-immunization) were cultured with 10 μM of MOG₃₅₋₅₅, 50 ng/ml IL-12 and 50 ng/ml IL-18 for 48 hours. Cells were expanded with IL-2 for 48 hours and then 1.3×10⁶ blasts adoptively transferred into naive mice. On the day of transfer and again 48 hours later, mice received 200 ng of pertussis toxin. Data shown are the mean disease score of recipient mice from 5 mice in each group.

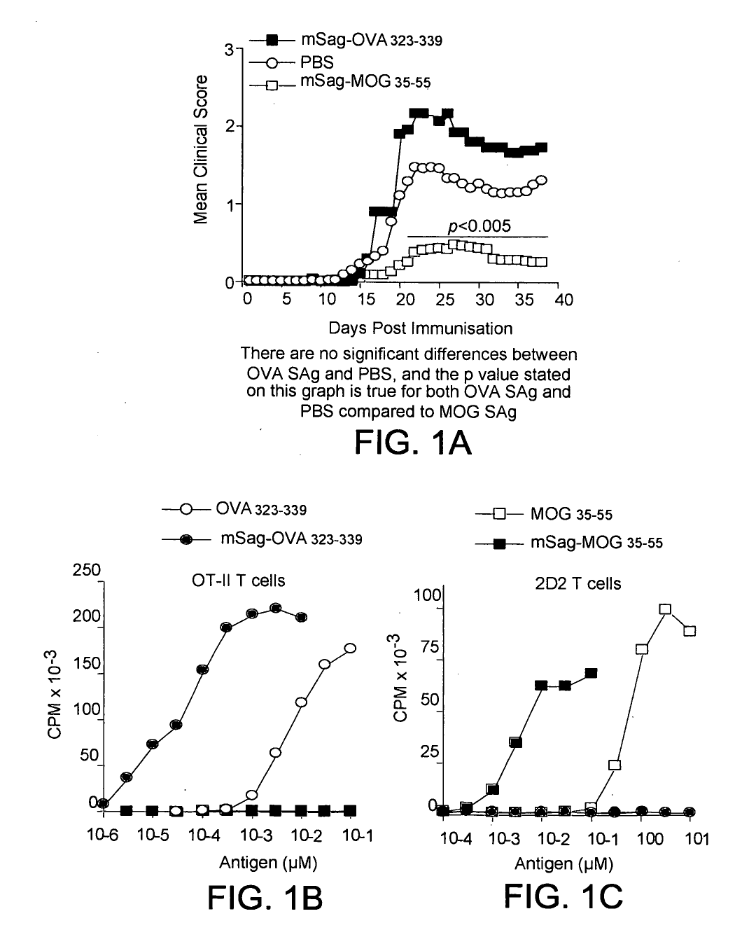

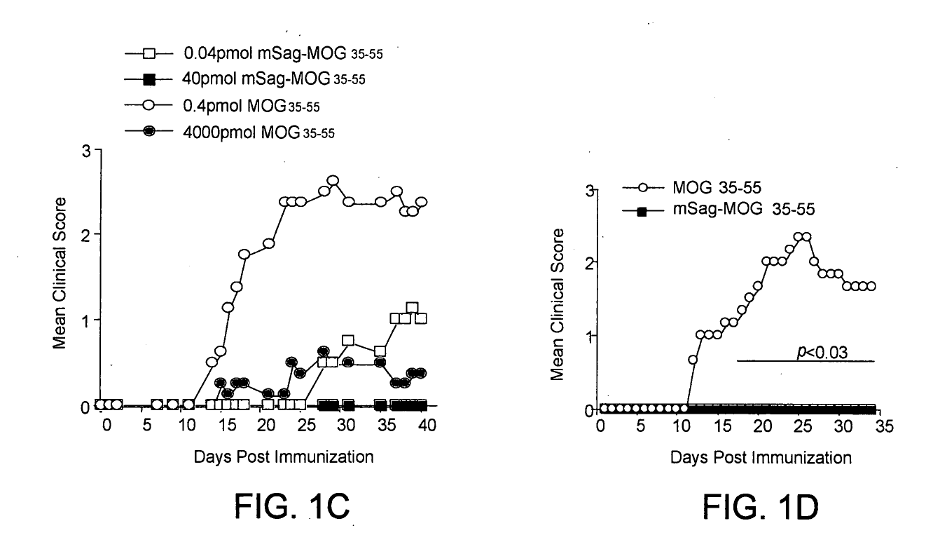

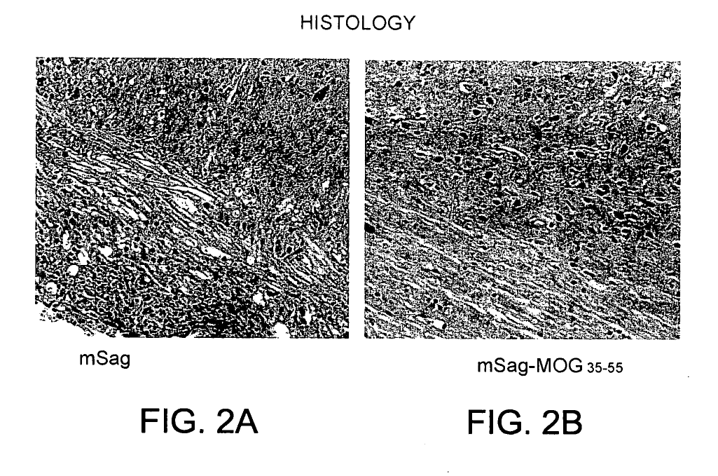

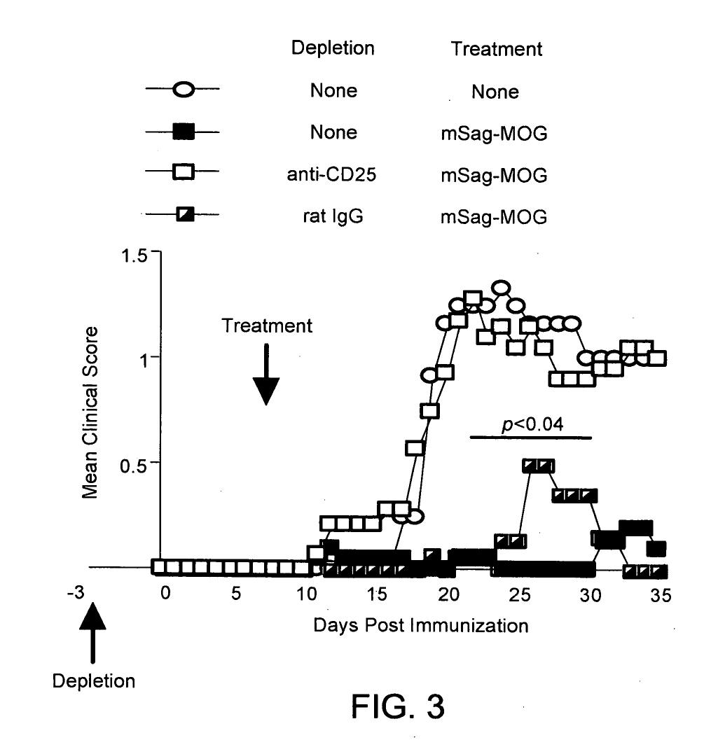

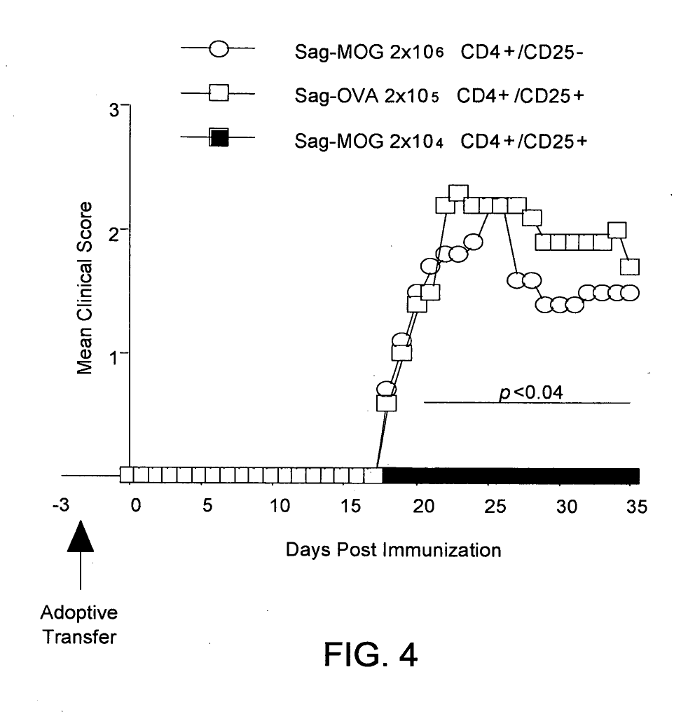

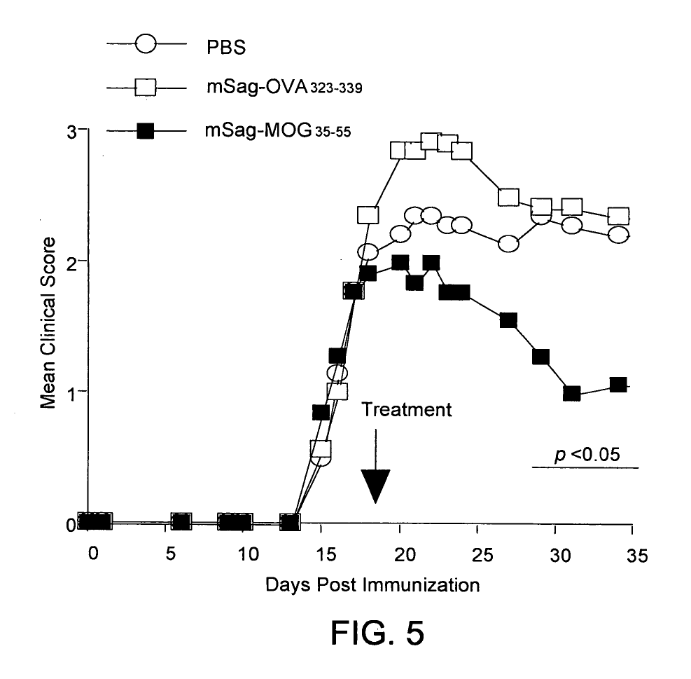

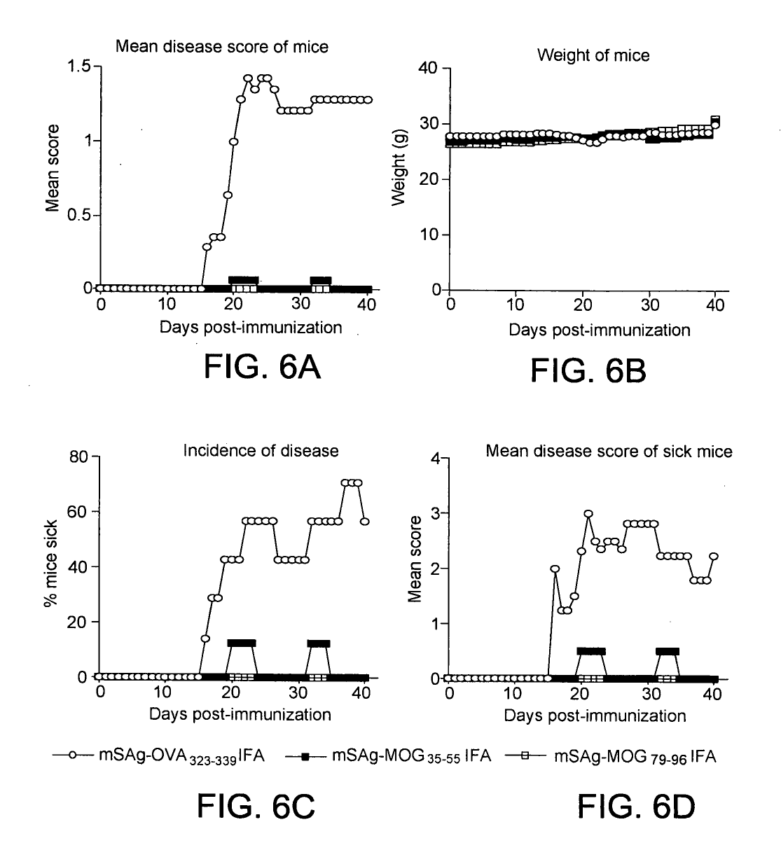

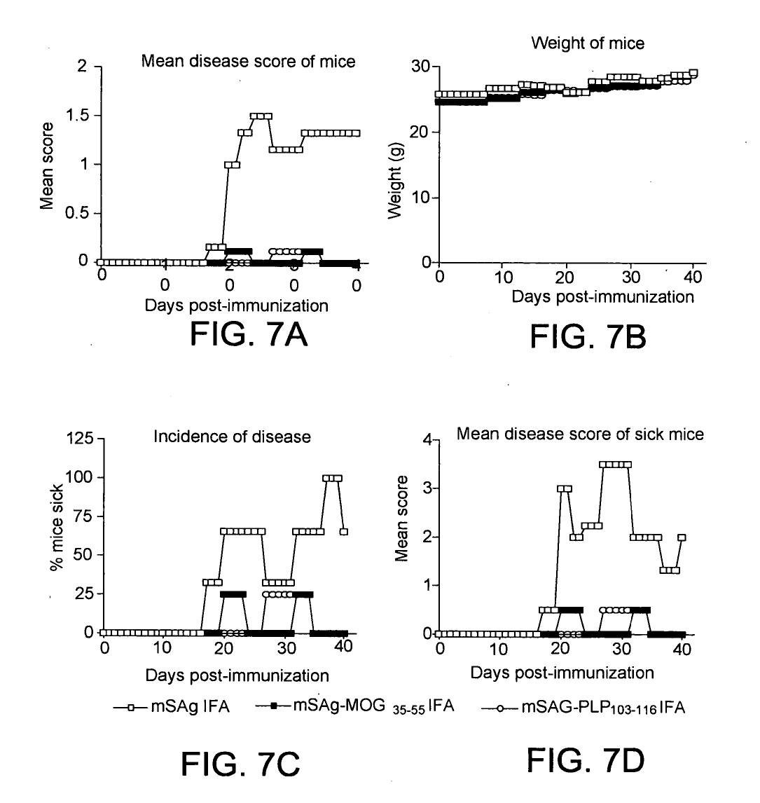

FIG. 2 illustrates that mSag-MOG₃₅₋₅₅ prevents infiltration or accumulation of cells in the CNS. Mice were immunized as in FIG. 1A and treated seven days later with mSag (A) or mSag-MOG₃₅₋₅₅ (B). Spinal cords were removed from mice with a disease score of 3 (A) and 0 (B) at day 40, fixed and embedded in paraffin. Six-micrometer sections were stained with hematoxylin and eosin. Representative sections from one of 3 experiments with similar results are shown. Original magnifications: 400×.

FIG. 3 illustrates that suppression of EAE by mSag-MOG₃₅₋₅₅ requires CD4⁺/CD25⁺ Treg cells. Mice were immunized and treated as in FIG. 1A. Indicated groups of mice were depleted of CD25+ cells using the mAb PC61 or injected with normal rat IgG three days before immunization. Mean disease scores of mice pooled from 4 independent experiments are shown.

FIG. 4 illustrates that CD4⁺/CD25⁺ Treg cells from mice treated with mSag-MOG₃₅₋₅₅ prevent the development of EAE. Mice were treated with 40 pmol mSag-MOG₃₅₋₅₅ or 40 pmol mSag-OVA₃₂₃₋₃₃₉. CD4⁺/CD25³¹ or CD4⁺/CD25⁺ cells were purified from draining lymph nodes 7 days later and the indicated numbers were injected into naive C57BL/6J mice. Recipient mice were immunized with MOG₃₅₋₅₅ one day later to induce EAE and data shown are the mean score of mice pooled from two independent experiments.

FIG. 5 illustrates that mSag-MOG₃₅₋₅₅ reduces the severity of EAE when given after disease onset. Mice were immunized as in FIG. 1A. At day 18, mice were divided into groups of similar disease score (˜2) treated with PBS or 40 pmol of mSag-MOG₃₅₋₅₅ or mSag-MOG₃₂₃₋₃₃₉. Mean disease score of mice are shown from one representative experiment of three performed

FIG. 6 illustrates that mSAg-MOG₃₅₋₅₅ and mSAg-MOG₇₉₋₉₆ can suppress EAE induced by MOG₃₅₋₅₅ Mice were immunized with 50 μg of MOG₃₅₋₅₅ and 500 μg heat killed M tuberculosis subcutaneously in the flank followed by 250 ng PTxn 24 hours later. Seven days after the immunization mice were treated with either 1 μg of mSAg-MOG₃₅₋₅₅, 1 μg mSAg-MOG₇₉₋₉₆, or 1 μg mSAg-OVA₃₂₃₋₃₃₉ emulsified in IFA. Mice were assessed daily for clinical signs of disease. Results shown are pooled from two experiments. Due to the sample size, the differences between animals treated with mSAg-MOG₃₅₋₅₅, mSAg-MOG₇₉₋₉₆ and mSAg-OVA₃₂₃₋₃₃₉ were only significant to a P value of <0.1.

FIG. 7 illustrates mSAg-MOG₃₅₋₅₅ and mSAg-PLP₁₀₃₋₁₁₆ can suppress EAE induced by MOG₃₅₋₅₅. Mice were immunized with 50 μg of MOG₃₅₋₅₅ and 500 μg heat killed M tuberculosis subcutaneously in the flank followed by 250 ng PTxn 24 hours later. Seven days after the immunization mice were treated with either 1 μg of mSAg-MOG₃₅₋₅₅, 1 μg mSAg-PLP₁₀₃₋₁₁₆, or 1 μg mSag emulsified in IFA. Mice were assessed daily for clinical signs of disease. Results shown are from one experiment. Due to the sample size, the differences observed between animals treated with mSAg-MOG₃₅₋₅₅, mSAg-PLP₁₀₃₋₁₁₆ and mSag were not significant.

### PREFERRED EMBODIMENT(S)

The following is a description of the present invention, including preferred embodiments thereof, given in general terms. The invention is further elucidated from the disclosure given under the heading “Example”, which provides experimental data supporting the invention and specific examples thereof.

The inventors have demonstrated that a single low dose of a T cell binding defective bacterial superantigen (mSag) coupled to a myelin oligodendrocyte glycoprotein (MOG) peptide (MOG₃₅₋₅₅) completely prevented the onset of experimental autoimmune encephalomyelitis (EAE) and also ameliorated symptoms of established EAE in mice. The results indicate the efficacy of using an immunomodulatory construct consisting of MOG or a MOG peptide coupled to a T cell binding defective bacterial superantigen in a therapeutic method to treat MS. The results also indicate the advantages of using a construct of the invention over administration of free encephalitogenic peptides.

The inventors have further demonstrated that constructs including a T cell binding defective bacterial superantigen (mSag) coupled to a MOG peptide (MOG₇₉₋₉₆), or coupled to a proteolipid protein (PLP) peptide (PLP₁₀₃₋₁₁₆), are able to suppress the development of MOG₃₅₋₅₅ induced EAE in mice. The inventors contemplate that this bystander effect is applicable to all myelin-associated proteins, and that it may allow for effective treatment of not only the initial or acute stages of MS, but also help suppress progression and relapse and treat late stages of the disease.

As used herein, the term “treatment” is to be considered in its broadest context. The term does not necessarily imply that subject is treated until total recovery. Accordingly, “treatment” broadly includes the amelioration of the symptoms or severity of a particular disorder, or preventing or otherwise reducing the risk of developing a particular disorder.

It will be appreciated by those of general skill in the art to which the invention relates, having regard to the nature of the invention and the result reported herein, that the present invention is applicable to a variety of different animals. Accordingly, a “subject” includes any animal of interest. In particular the invention is applicable to mammals, more particularly humans.

Myelin-Associated Proteins and Peptides

Any myelin-associated protein or peptide may be used in the present invention. A “myelin-associated protein” is any protein which forms a part of the myelin sheath surrounding nerves cells. Exemplary myelin-associated proteins include myelin oligodendrocyte glycoprotein (MOG), myelin-associated glycoprotein (MAG), proteolipid protein (PLP), myelin basic protein (MBP), and Myelin oligodendrocyte glycoprotein (MOG); Oligodendrocyte-specific glycoprotein (OSP); 2′,3′-Cyclic nucleotide 3′ phosphodiesterase (CNPase); Myelin-associated oligodendrocytic basic protein (MOBP); α-B Crystallin (αB-C); S100β Protein; Transaldolase-H (Tal-H); and Oligodendrocyte myelin glycoprotein (OMGP).

In a preferred embodiment of the invention the myelin-associated peptide is MOG, PLP or MBP.

Amino acid and nucleic acid sequence information for each of the above myelin-associated proteins can be found and/or are accessible to those skilled in the art. It should be appreciated that myelin-associated proteins or peptides from organisms other than humans may be used in the present invention, provided they are compatible with the animal to be treated.

A “peptide” of use in the invention is any peptide forming a part of a myelin-associated protein. Preferably the peptide is an immunogenic peptide, that is, a peptide which is capable of eliciting an immune response in humans, i.e. peptides with the capacity to bind to any human leukocyte antigen (HLA). Skilled persons may appreciate peptides of use in the invention on the basis of information regarding peptides implicated in development of MS, or information regarding peptides which have been used in attempts to treat MS. By way of example, the following peptides may be of use in the invention: MBP; 13-32, 83-99, 111-129 and 145-170, PLP; 30-49, 40-60, 89-106, 95-116, 104-117, 139-151, 142-153, 178-191, 184-199, 190-209, MOG; 1-22, 11-30, 21-40, 31-50, 34-56, 63-87, 64-96, 71-90, 146-154, MAG; 596-612, 609-626, CNPase; 343-373, MOBP; 21-39, OSP; 52-78, αB-C; 21-40, 41-60, 131-150, S100β Protein; 76-91²². Further myelin-associated peptides are listed in WO 95/06727, W096/34622, WO03/033645, WO01/31037, WO 97/35879 and WO95/07096. Peptides of particular use in the invention include MBP, PLP, and MOG related peptides which show immunodominance.

Pharmaceutically acceptable salts of proteins and peptides as well as stereoisomers of proteins and peptides are suitable for use in the present invention. Further, a protein or peptide of the invention may be composed of L-amino acids, D-amino acids or a mixture thereof.

A protein or peptide of use in the invention may be purified from natural sources, or preferably derived by chemical synthesis (for example, FMOC solid phase peptide synthesis as described in Fields G B, Lauer-Fields J L, Liu R Q and Barany G (2002) Principles and Practice of Solid-Phase peptide Synthesis; Grant G (2002) Evaluation of the Synthetic Product. Synthetic Peptides, A User's Guide, Grant G A, Second Edition, 93-219; 220-291, Oxford University Press, New York) or genetic expression techniques, methods for which are readily known in the art to which the invention relates. The inventors contemplate production of a protein or peptide of the invention by an appropriate transgenic animal, microbe, or plant.

The invention provides a treatment of multiple sclerosis, comprising a T cell binding defective superantigen coupled to one or more myelin-associated protein, peptide or functionally equivalent variant thereof, or a pharmaceutical composition comprising same.

Functionally Equivalent Variants

Use of functionally equivalent variants of myelin-associated proteins or peptides may be made. “Functionally equivalent variants” are those molecules capable displaying at least a degree of the functional characteristics of a native myelin-associated protein or peptide. In the context of the present invention functionally equivalent variants will have an ability to stimulate T cells in an antigen-specific manner. For example, an antibody specific for a TCR recognizing a myelin-derived peptide, or any other protein which could activate T cells in an antigen-specific manner. The variants will also need to retain the ability to be effectively coupled to a mSag in accordance with the invention.

“Functionally equivalent variants” should be taken to include allelic variants of any published myelin-associated protein or peptide and other variant polypeptides. In one embodiment a functionally equivalent variant protein or peptide will have at least 80% sequence identity with a native myelin-associated protein or peptide, at least 90%, at least 95% or at least 99% sequence identity with a native myelin-associated protein or peptide. The variant polypeptide may be one in which conservative amino acid substitutions have been made compared to the published amino acid sequence data for a particular protein. Persons of general skill in the art to which the invention relates will appreciate appropriate conservative amino acid changes or substitutions having regard to established rules in this regard.

In addition, a functionally equivalent variant protein or peptide may be one which shows molecular mimicry with an antigenic myelin-derived protein or peptide (i.e. the overall sequence similarity is low, but contains amino acids necessary for binding to HLA molecules and the TCR) and can therefore stimulate T cells cross-reactive with myelin-derived proteins or peptides.

“Functionally equivalent variants” also include myelin-associated proteins or peptides which have been extended by, or fused to, heterologous amino acid motifs or sequences where desired.

The term “functionally equivalent variants” should also be taken to include polypeptides which have been chemically modified. For example peptides may be modified by acetylation, glycosylation, cross-linking, disulfide bond formation, cyclization, branching, phosphorylation, conjugation or attachment to a desirable molecule, acylation, ADP-ribosylation, amidation, covalent attachment of a lipid or lipid derivative, covalent attachment of phosphotidylinositol, demethylation, formation of covalent cross-links, formation of cysteine, formation of pyroglutamate, formylation, gamma-carboxylation, GPI anchor formation, hydroxylation, methylation, myristoylation, oxidation, pegylation, proteolytic processing, prenylation, racemization, conversion from L-isomer to D-isomer, sulfation, or otherwise to mimic natural post-translational modifications, or to aid in presentation for example. The peptides may also be modified to include one or more non-naturally occurring amino acids, as will be known in the art. Amino acids of a peptide may also be modified by substitution of R groups for other chemical groups as may be known in the art. In addition, amino acids may be substituted with chemical groups which mimic them; for example, benzimidazole is a known mimic of R and 1,4-benzodiazepine a mimic of G-D (see Curr Protein Pept Sci 2005 Apr.; 6(2):151-169. Peptides of the invention may also be modified by arrangement of amino acid groupings from the peptide on a non-peptide scaffold. Considerations for designing such modified peptides are discussed in Curr Protein Pept Sci. 2005 Apr.; 6(2):151-169 (Sillerud and Larson).

An example of a functionally equivalent variant includes glatiramer acetate (for example, the product Copaxone® (Teva Pharmaceutical Industries Ltd, Israel), CAS- 147245-92-9). Glatiramer acetate consists of the acetate salts of synthetic polypeptides, containing four naturally occurring amino acids: L-glutamic acid, L-alanine, L-tyrosine, and L-lysine with an average molar fraction of 0.141, 0.427, 0.095, and 0.338, respectively. Glatiramer acetate is designated L-glutamic acid polymer with L-alanine, L-lysine and L-tyrosine, acetate (salt).

Functionally equivalent variants of a myelin-associated protein or peptide may be readily identified using standard methodology having regard to the description of the invention described herein. By way of example, the compatibility of a functionally equivalent variant with the present invention may be tested using in vitro proliferation assays such as stimulation of T cells from transgenic mice expressing a TCR with specificity against myelin-related peptide with functionally equivalent variants. Thus any variant peptide of a myelin associated protein which stimulates T cells may be acceptable.

Superantigens

The invention makes use of T cell binding defective superantigens (which may be referred to herein as “mSag”). “T cell binding defective superantigens” are those molecules which are structurally a superantigen, including an intact MHC binding site, but have a disrupted T-cell receptor binding site which is not fully functional. The molecule thus has reduced, and preferably little or no ability to activate T cells.

A superantigen is a secreted bacterial globular protein that possesses one larger domain of the b-grasp fold and a smaller domain of the OB-fold type. The molecule has one and sometimes two binding sites for the Major Histocompatibility Complex Class II molecule and a third binding site specific for the V beta domain of the T cell Receptor. Superantigen activity is recognised as potent stimulation of T cells all of which have common T cell Receptor V beta domains. A modified superantigen for the purpose of this description is one in which the T cell Receptor V beta binding has been disrupted so that the binding affinity between superantigen and all T cell Receptors that it normally binds is reduced. Most preferably, the T cell stimulation activity is reduced by at least approximately 10,000 fold. That is to say, to produce the same level of T lymphocyte stimulation 10,000 times more superantigen is required compared to the wild-type superantigen. The reduction in TCR binding can be measured by the reduced ability of the modified superantigen to stimulate T lymphocytes in culture, for example peripheral blood lymphocytes.

For the purpose of this invention, modification of the TCR binding site of the superantigen by necessity does not alter the MHC class II binding site of the superantigen which is in a separate and distinct location, so that the modified superantigen binds with substantially the same affinity to MHC class II bearing antigen presenting cells as the wild-type superantigen.

Persons of skill in the art to which the invention relates may appreciate various wild type superantigens upon which the invention can be based. However, by way of example, superantigens of the bacterial species Staphylococcus aureus and Streptococcus pyogenes are of particular use. Specific superantigens of use in the invention include: Any allelic variant of the SMEZ family which includes all allelic variants from SMEZ-1 to SMEZ-39 or those showing greater than 90% nucleotide homology to any SMEZ alleles included in this family of closely associated alleles; and SPE-C, SEA. Details of these superantigens, including exemplary nucleotide sequences are provided in GenBank FASTA file under the accession numbers
SMEZ-2 >gi|5814012|gb|AF086626.1|AF086626
SMEZ-3 gi|46093519|dbj|AB046865.1 |[46093519]
SMEZ-4 gi|7672824|gb|AF143654.1|AF143654[7672824]
SMEZ-5 gi|7672826|gb|AF143655.1|AF143655[7672826]
SMEZ-6 gi|7672828|gb|AF143656.1|AF143656[7672828]
SMEZ-7 gi|7672829|gb|AF143657.1|AF143657[7672829]
   through to
SMEZ-39 gi|62912395|gb|DQ001539.1|[62912395]
SEA gi|153120|gb|M18970.1|STATOXAA S. aureus enterotoxin A (entA) gene

Furthermore, GenBank accession numbers to the corresponding amino acid sequences are provided below for SEA, SPE-C and SMEZ-2 as a representative of the SMEZ family

>gi|153121|gb|AAA26681.1|staphylococcal enterotoxin A precursor

>gi|153121|gb|AAA26681.1|staphylococcal entero-
toxin A precursor
MKKTAFTLLLFIALTLTTSPLVNGSEKSEEINEKDLRKKSELQGTALGNL
KQIYYYNEKAKTENKESHDQFLQHTILFKGFFTDHSWYNDLLVDFDSKDI
VDKYKGKKVDLYGAYYGYQCAGGTPNKTACMYGGVTLHDNNRLTEEKKVP
INLWLDGKQNTVPLETVKTNKKNVTVQELDLQARRYLQEKYNLYNSDVFD
GKVQRGLIVFHTSTEPSVNYDLFGAQGQYSNTLLRIYRDNKTINSENMHI
DIYLYTS
>gi|153821|gb|AAA27017.1|streptococcal pyrogenic
exotoxin type C (speC) precursor
MKKINIIKIVFIITVILISTYFTYHQSDSKKDISNVKSDLLYAYTITPYD
YKDCRVNFSTTHTLNIDTQKYRGKDYYISSEMSYEASQKFKRDDHVDVFG
LFYILNSHTGEYTYGGITPAQNNKVNHKLLGNLFISGESQQNLNNKIILE
KDIVTFQEIDFKIRKYLMDNYKIYDATSPYVSGRIEIGTKDGKHEQIDLF
DSPNEGTRSDIFAKYKDNRIINMKNFSHFDIYLEK
>gi|5814013|gb|AAD52087.1|AF086626_1 mitogenic
exotoxin Z 2 precursor
LEVDNNSLLRNIYSTIVYEYSDIVIDFKTSHNLVTKKLDVRDARDFFINS
EMDEYAANDFKTGDKIAVFSVPFDWNYLSKGKVTAYTYGGITPYQKTSIP
KNIPVNLWINGKQISVPYNEISTNKTTVTAQEIDLKVRKFLIAQHQLYSS
GSSYKSGRLVFHTNDNSDKYSFDLFYVGYRDKESIFKVYKDNKSFNIDKI
GHLDIEIDS

For the purposes of this specification and claims, reference to “SMEZ” should be taken as being a reference to any member of the SMEZ family as referred to above.

The inventors have found that an immunomodulatory construct based on SMEZ-2 to be of particular use in the invention.

The T-cell receptor binding site of an appropriate wild type superantigen can be modified by a number of means to remove or minimize TCR binding. For example, mutations may be created in a wild-type superantigen by means known in the art. A preferred method involves the use of overlap PCR. As it will be appreciated by skilled persons, this technique generally involves the use of two complimentary overlapping synthetic primers which hybridise to specific sequences in the wild-type gene template contained in a bacterial plasmid in which the specific mutation is to be introduced. The primers contain a mutation within the overlap region. Extension of the primers allows for the synthesis of a new nucleic acid strand containing the specific mutation. The mutated nucleic acid plasmid is then introduced into bacteria to produce the recombinant mutant protein. Specific examples of techniques which may be used to arrive at appropriate modified superantigen of use in the present invention are provided in WO 03/101173 and WO 02/45739.

Table 1 provides examples of specific modified superantigens of use in the present invention. * indicates a particularly preferred modified superantigen.

TABLE 1
Mutant

SMEZ-2 W75L
SMEZ-2 D42C
SMEZ-2 W75L.D42C.K182Q*
SMEZ-2 Y18A
SMEZ-2 W75L.D42C.K182Q.Y18A
*indicates a particularly preferred modified superantigen.

Immunomodulatory Construct

The coupling of the mSag to a myelin-associated protein, peptide or functionally equivalent variant thereof (the antigen) can be accomplished by using any number of techniques standard in the art. It should be appreciated that the term “coupling” is intended to include any means by which the mSag may be associated with one or more antigen, and includes fusion, chemical linkage and the like. For example, the constructs may be produced recombinantly in the form of fusion proteins with the antigen formed as a genetic extension of either the C-terminus or N-terminus of the mSag. Special consideration is preferably made to ensure the coupled antigen does not interfere with MHC class II binding. Alternatively, the antigen and the mSag may be coupled in accordance with techniques described herein after under the heading “Examples” or as described in WO 02/45739.

By way of a general example, the mSag may be coupled to an antigen using succinimidyl-S-acetylthiopropionate (SATP, Pierce Chemicals). In a first step the antigen undergoes a thiolation process using SATP. The antigen is first dissolved in NaPO₄ whilst SATP in dissolved in dimethysulfoxide (DMSO). The SATP solution is added to the protein at a molar ratio of 1:1 and incubated at room temperature for 2 hours. Unbound SATP is removed by passing through VIVASPIN concentrator (5 kDa size exclusion), at about 10,000 rpm for about 5 minutes. NaPO₄, is added and repeated 5 times. Alternatively the antigen is passed through a column of sephadex G25 desalting column. Immediately after removal of free SATP the sulphydryl group of antigen-SATP is deprotected by a deacetylation process. This may be achieved by using a deacetylation solution including NaPO₄, EDTA, and Hydroxylamine-HCl. The mSag is then added to the solution in a 1:10 molar ration of mSag to protein-SATP. A coupling buffer may also be added as will be readily known in the art. The mSag and protein-SATP are then incubated at room temperature for about 2 hours.

If the antigen has a naturally occurring cysteine residue, coupling to a mSag may be achieved directly without the need to introduce a reactive sulphydryl group. In this case, coupling would follow the established procedure outlined above. If the antigen does not have a naturally occurring cysteine, a number of standard procedures may be used to introduce a reactive sulphydryl group. By way of example, a cysteine residue can be introduced genetically into a recombinant peptide and the polypeptide expressed from a heterologous expression system (prokaryotic or eukaryotic). It will be readily appreciated by those skilled in the art that the site of introducing the reactive sulphydryl group is preferably exposed on the surface of the molecule. Skilled persons will readily be able to identify such residues on the basis of actually or putative 3D protein structure.

If a peptide is the chosen antigen, then this can be chemically synthesized with the addition of a cysteine on the N-terminus or C-terminus and the coupled directly to the exposed cysteine by oxidation reaction.

By way of further example, recombinant constructs of the invention may be constructed by extending the C-terminus of the mSag by creating a flexible linker of 5 to 10 glycine residues in a genetic construct according to standard methodology. Furthermore a nucleic acid construct may be provided which includes a multi-cloning site to allow the addition of selected antigen or peptide gene sequences. The entire construct may then be expressed to create a recombinant fusion construct protein.

By way of another example, an alternative coupling process may include the use of Streptavidin coupled to a mSag. Streptavidin binds 4 molecules of biotin tightly. This creates a mSag streptavidin fusion construct that allows the multivalent attachment of biotinylated proteins and peptides. In accordance with standard methodology, an antigen is biotinylated then added to the mSag streptavidin fusion construct to form the final conjugate. Such coupling may be completed in accordance with techniques readily known in the art.

In a preferred embodiment of the invention the coupling between the mSag and the antigen is reversible. What is particularly preferred is that the mSag is capable of releasing the immunomodulatory antigen so that it is correctly presented by the APC. It will be recognised by persons of skill in the art to which the invention relates that the release of the antigen from the construct may be achieved by intracellular or intralysosomal enzymatic cleavage. This process may be assisted by introducing the appropriate proteolytic site into the coupling region of the construct. The release may also be achieved by chemical means, which includes oxidation/reduction reactions involving disulphides and free sulphydryl groups. This process may also be assisted by introducing into the coupling region certain amino acid residues, eg. cysteine. Persons of general skill in the art will readily appreciate techniques by which such cleavage or release sites may be incorporated into the constructs. However, by way of example, site directed mutagenesis, recombinant cloning and/or expression, chemical synthesis of peptides or nucleic acids coding therefore, which include appropriate sites.

Zhong et al, have shown that a multi-antigen of encephalitogenic MBP, PLP, and MOG peptides effectively can suppress disease in EAE²⁴. Hence coupling need not be limited to individual peptides. Because immune responses to peptides are tightly restricted by the MHC polymorphisms of the host, it may be appropriate in some circumstances, to immunize with sets of peptides to generate broad spectrum immunomodulatory agents. Multiple peptides representing various components of a larger antigen may be coupled by procedures described above or modified versions therefor which would be clear to those skilled in the art, to provide a mixed antigen:mSag-based construct. Moreover, the ratio of peptides could be easily controlled to fine tune the immune response to a more desired outcome.

The purification of the constructs of the invention may be performed by any appropriate means. Those of general skill in the art to which the invention relates will readily appreciate appropriate techniques having regard to the nature of the construct and the method by which it may have been generated. However, by way of example, constructs of the invention may be purified by HPLC and/or by size exclusion chromatography using a Superose12 column or any other suitable chromatography media that allows separation of proteins on the basis of size.

Nucleic Acid Vectors or Constructs

To the extent that proteins, peptides, mSAG and the immunomodulatory constructs may be produced by recombinant techniques the invention provides nucleic acids encoding proteins, peptides, mSAG and immunomodulatory constructs of the invention, as well as and constructs or vectors which may aid in the cloning and expression of such nucleic acids.

Those of general skill in the art to which the invention relates will readily be able to identify nucleic acids which encode proteins or peptides of use in the invention, including desired fusion peptides or proteins, on the basis of the amino acid sequences of the desired proteins or peptides, any sequence information contained herein and in public databases, the genetic code, and the understood degeneracy therein.

Nucleic acid constructs or vectors will generally contain heterologous nucleic acid sequences; that is nucleic acid sequences that are not naturally found adjacent to the nucleic acid sequences of the invention. The constructs or vectors may be either RNA or DNA, either prokaryotic or eukaryotic, and typically are viruses or a plasmid. Suitable constructs are preferably adapted to deliver a nucleic acid of the invention into a host cell and are either capable or not capable of replicating in such cell. Recombinant constructs comprising nucleic acids of the invention may be used, for example, in the cloning, sequencing, and expression of nucleic acid sequences of the invention.

Those of skill in the art to which the invention relates will recognise many constructs suitable for use in the present invention. However, the inventors contemplate the use of cloning vectors such as pUC and pBluescript and expression vectors such as pCDM8, adeno-associated virus (AAV) or lentiviruses to be particularly useful.

The constructs may contain regulatory sequences such as promoters, operators, repressors, enhancers, termination sequences, origins of replication, and other appropriate regulatory sequences as are known in the art. Further, they may contain secretory sequences to enable an expressed protein to be secreted from its host cell. In addition, expression constructs may contain fusion sequences (such as those that encode a heterologous amino acid motif) which lead to the expression of inserted nucleic acid sequences of the invention as fusion proteins or peptides.

In accordance with the invention, transformation of a construct into a host cell can be accomplished by any method by which a nucleic acid sequence can be inserted into a cell. For example, transformation techniques include transfection, electroporation, microinjection, lipofection, adsorption, and biolistic bombardment.

As will be appreciated, transformed nucleic acid sequences of the invention may remain extrachromosomal or can integrate into one or more sites within a chromosome of a host cell in such a manner that their ability to be expressed is retained.

Any number of host cells known in the art may be utilised in cloning and expressing nucleic acid sequences in accordance with the invention. For example, these include but are not limited to microorganisms such as bacteria transformed with recombinant bacteriophage DNA, plasmid DNA or cosmid DNA expression vectors; yeast transformed with recombinant yeast expression vectors; insect cell systems infected with recombinant virus expression vectors (e.g., baculovirus); animal cell systems such as CHO (Chinese hamster ovary) cells using the pEE14 plasmid system; plant cell systems infected with recombinant virus expression vectors (e.g., cauliflower mosaic virus, CaMV; tobacco mosaic virus, TMV) or transformed with recombinant plasmid expression vectors (e.g., Ti plasmid).

A recombinant protein or peptide in accordance with the invention may be recovered from a transformed host cell, or culture media, following expression thereof using a variety of techniques standard in the art. For example, detergent extraction, sonication, lysis, osmotic shock treatment and inclusion body purification. The protein may be further purified using techniques such as affinity chromatography, ion exchange chromatography, filtration, electrophoresis, hydrophobic interaction chromatography, gel filtration chromatography, and chromatofocusing.

As mentioned herein before, the immunomodulatory constructs of the invention may be produced in the form of a fusion protein between the superantigen gene sequence and the gene sequence coding for the antigen of interest. This might be expressed as either a C-terminal fusion, where the antigenic sequence is joined to the 3′ end of the superantigen sequence, or as an N-terminal fusion where the gene coding for the antigen is joined to the 5-end of the superantigen gene. Means for generating fusion proteins are known in the art to which the invention relates, and include chemical synthesis and recombinant techniques. The inventors contemplate Strep-tag (Sigma-Genosys), Impact™ system (New England Biolabs), his-tag, and the eg pMAL™-p2 expression system (New England BioLabs), to be particularly useful in producing fustion proteins. In addition, fusion tags of use in recombinant protein expression and purification have been described by R. C. Stevens. “Design of high-throughput methods of protein production for structural biology” Structure, 8, R177-R185 (2000).

Pharmaceutical Compositions

In addition to the immunomodulatory constructs, the invention also provides pharmaceutical compositions containing them along with one or more pharmaceutically acceptable carriers, adjuvants, excipients and/or diluents.

As-used herein, the phrase “pharmaceutically acceptable diluents, carriers and/or excipients” is intended to include substances that are useful in preparing a pharmaceutical composition, may be co-administered with the immunomodulatory constructs or nucleic acids of the invention while allowing them to perform their intended function, and are generally safe, non-toxic and neither biologically nor otherwise undesirable. Pharmaceutically acceptable diluents, carriers and/or excipients include those suitable for veterinary use as well as human pharmaceutical use. Examples of pharmaceutically acceptable diluents, carriers and/or excipients include solutions, solvents, dispersion media, delay agents, emulsions and the like.

Those skilled in the art will readily appreciate that a variety of acceptable pharmaceutical carriers, adjuvants, excipients and/or diluents may be employed in preparing a composition of the invention. As will be appreciated the choice of acceptable pharmaceutical carriers, adjuvants, excipients and/or diluents, may be dictated to some extent by the intended dosage form of the composition and the mode of administration of the composition. By way of example, suitable carriers, excipients, and diluents include alum and TitreMax.

In respect of adjuvants the choice of adjuvant may differ depending on a number of variables. By way of example, the chemical nature of the adjuvant and its mode of action in a particular subject, the level of adjuvancy required, the observed side effects of a particular adjuvant, the nature of the antigen used, the tolerance of a particular subject to an adjuvant, which species of animal the subject belongs to, and the age and/or general health of a subject. Examples of suitable adjuvants may include: oil-based adjuvants (for example, Freunds complete or incomplete, mineral oil, emulsified peanut oil (adjuvant 65), paraffin and vegetable oils), liposomes, mineral compounds, aluminum hydroxide, aluminum phosphate, calcium phosphate, endotoxins, cholesterol, fatty acids, aliphatic amines, monophosphoryl lipid A, immunostimulating complexes (ISCOMs) (for example ISCOMs with Quil-A), and Syntex adjuvant formulations (SAFs) containing the threonyl derivative or muramyl dipeptide.

In addition to standard diluents, carriers, adjuvants and/or excipients, a pharmaceutical composition in accordance with the invention may be formulated with additional constituents, or in such a manner, so as to enhance the activity of a construct, or nucleic acid of the invention, or help protect the integrity of such agents. For example, the composition may further comprise constituents which provide protection against proteolytic degradation, enhance bioavailability, decrease antigenicity, or enable slow release upon administration to a subject. For example, slow release vehicles include macromers, poly(ethylene glycol), hyaluronic acid, poly(vinylpyrrolidone), or a hydrogel.

Additionally, it is contemplated that a pharmaceutical composition in accordance with the invention may be formulated with additional active ingredients which may be of benefit to a subject in particular instances. Persons of ordinary skill in the art to which the invention relates will readily appreciate suitable additional active ingredients having regard to the description of the invention herein and the nature of the disease or symptoms to be treated, for example. By way of example, a pharmaceutical composition in accordance with the present invention may include a Copaxone®, or a combination of Copaxone® and the interferons such as Betaferon®, Beta interferon-1A (Avonex®, Rebif®).

Compositions of the invention may be produced by a number of techniques standard in the art by mixing the individual ingredients with one or more diluents, carriers, adjuvants and/or excipients.

Compositions of the invention may be converted to customary dosage forms such as solutions, orally administrable liquids, injectable liquids, tablets, coated tablets, capsules, pills, granules, suppositories, trans-dermal patches, suspensions, emulsions, sustained release formulations, gels, sprays (atmosier or aerosol), powders and immunoliposomes. Additionally, sustained release formulations may be utilised. The dosage form chosen will reflect the mode of administration desired to be used. Particularly preferred dosage forms include orally administrable tablets, gels, pills, capsules, semisolids, powders, sustained release formulation, suspensions, elixirs, aerosols, ointments or solutions for topical administration, and injectable liquids. Skilled persons will readily appreciate alternative dosage forms.

Kits

The constructs of the invention may be used in kits suitable for treatment of MS. Such kits will comprise at least construct of the invention in a suitable container. The construct may be formulated in a pharmaceutical composition ready for direct administration to a subject. Alternatively, the kit may comprise the construct in one container and a pharmaceutical carrier composition in another; the contents of each container being mixed together prior to administration. The kit may also comprise additional agents and compositions in further separate containers as may be necessary for a particular application. Further, kits of the invention can also comprise instructions for the use and administration of the components of the kit.

Any container suitable for storing and/or administering a pharmaceutical composition may be used in a kit of the invention. Suitable containers will be appreciated by persons skilled in the art. By way of example, such containers include vials and syringes. The containers may be suitably sterilised and hermetically sealed.

Methods of Treatment

The present invention also pertains to methods for the treatment of multiple sclerosis comprising at least the step of administering to a subject in need thereof a therapeutically effective amount of an immunomodulatory construct of the invention. Such a method may comprise the administration of a pharmaceutical composition of the invention.

In light of the bystander effect demonstrated by the inventors, the method may involve the administration of an immunomodulatory construct of the invention which includes one or more myelin-associated protein, peptide or functionally equivalent variant thereof which is distinct from one or more myelin-associated protein or peptide which is at the origin of the multiple sclerosis; ie the myelin-associated protein or peptide to which the disease-inducing immune cells are specific for.

A method of treating multiple sclerosis induced by a first myelin-associated protein or peptide, wherein the method includes at least the step of administering to a subject in need thereof an immunomodulatory construct.

In a related embodiment of the invention the method of treatment is an ex vivo method involving collecting peripheral blood mononuclear cells (PBMC) from MS patients, purification of CD4⁺/CD25^{HIGH} regulatory T cells and CD11c expressing dendritic cells. The purification of the cells involves standard methods using magnetic beads, such as Dynal® or Miltenyi® beads. The regulatory T cells are then stimulated by the superantigen conjugated with myelin-derived peptides, in a similar way as described for mouse cells²¹. Direct expansion of functional CD25+ CD4+ regulatory T cells by antigen-processing dendritic cells²³. Expanded regulatory T cells are then administered intravenously back to the same patients 1 to 2 weeks later.

As used herein, a “therapeutically effective amount”, or an “effective amount” is an amount necessary to at least partly attain a desired response.

The inventors contemplate administration by any means capable of delivering the active agents of the invention to a subject. By way of example, agents and compositions of the invention may be administered by one of the following routes: oral, topical, systemic (eg. transdermal, intranasal, or by suppository), parenteral (eg. intramuscular, subcutaneous, or intravenous injection), by administration to the CNS (eg. by intraspinal or intracisternal injection); by implantation, and by infusion through such devices as osmotic pumps, transdermal patches, and the like. Further examples may be provided herein after. Skilled persons may identify other appropriate administration routes.

Currently the most effective mode of administration of the immunomodulatory conjugate, in the EAE animal model of MS, is with an oil/water emulsion (incomplete Freund's adjuvant, IFA). An alternative formulation may include the substitution of IFA with MF59* as an adjuvant. Other adjuvants such as monophosphoryl lipid A, Quil-A, and Syntex adjuvant formulations may be exchanged for IFA²⁶.

As will be appreciated, the dose of an agent or composition administered, the period of administration, and the general administration regime may differ between subjects depending on variables such as the severity of symptoms of a subject, the nature of disorder to be treated, the mode of administration and dosage form chosen, tolerance to the agents administered, and the age, sex and/or general health of a subject.

Data obtained from cell culture assays and animal studies can be used in formulating a range of dosages for use in humans. The dosage may vary within this range depending upon the dosage form employed and the route of administration utilized. For any compound used in the method of the invention, the therapeutically effective dose can be estimated initially from animal models. Such information can be used to more accurately predetermine useful doses in humans.

### EXAMPLE

mSag-MOG₃₅₋₅₅

Materials and Methods

Mice

C57BL/6J mice were originally purchased from the Jackson Laboratory (Bar Harbour, Me.). The 2D2 transgenic mice express a TCR specific for fragment 35-55 of the MOG protein presented by IA^{b}. The generation of these mice has been described previously¹⁰. OTII mice with a transgenic TCR specific for fragment 323-339 of OVA presented by IA^{b} and were originally obtained from Dr. William Heath (Walter and Eliza Hall Institute for Medical Research, Melbourne, Australia). B6.SJL-PtprcaPep3b/BoyJArc (Formerly B6.Ly5.1 or CD45.1) congenic mice were purchased and imported from the Animal Resources Centre (ARC) Australia.

All mice were maintained by the Biomedical Research Unit, Malaghan Institute of Medical Research, Wellington, New Zealand. Experimental protocols were approved by the Wellington School of Medicine Animal Ethics Committee and performed according to the guidelines of the University of Otago. In all EAE experiments, sex and age matched mice were used between 8-12 weeks of age.

Peptides

The C-MOG₃₅₋₅₅ peptide used was >81% pure and corresponds to amino acid residues 35-55 of the mouse MOG sequence, with an N terminal cysteine residue added to enable coupling to the mSag (CMEVGWYRSPFSRVVHLYRNGK). The C-OVA₃₂₃₋₃₃₉ peptide used was >95% pure and corresponds to amino acid residues 323-339 of the mouse OVA sequence, with an N terminal cysteine residue added to enable coupling to the mSag (CISQAVHAAHAEINEAGR). Both peptides were synthesized by Mimotopes (Clayton, Australia).

Generation of the Modified Superantigen

Cloning and expression of the streptococcal mitogenic exotoxin Z-2 (SMEZ-2) from the Streptococcus pyogenes strain 2035 has been described previously¹¹. Residues essential for TCR binding were identified and selective mutations to abrogate TCR binding and to introduce a solvent exposed cysteine into the defective TCR binding site were created in the SMEZ-2 gene by PCR overlap¹¹. The final mutant gene SMEZ-2 M1 (SMEZ-2 W75L.D42C.K182Q) was expressed as a GST fusion protein from the pGEX-3C expression vector as previously described¹¹. Cultures were grown at 28° C. in terrific broth, and induced for 3-4 h after adding 0.1 mM isopropyl-β-D-thiogalactopyranoside (IPTG). Two-step purification involved affinity chromatography using glutathione agarose, cleavage with 3C protease, and finally cation exchange chromatography using carboxy methyl sepharose (Amersham Pharmacia, UK) as described previously¹¹. The general process is also described in WO 03/101173.

In an alternative process, SMEZ-2 M1 (SMEZ-2 W75L.D42C.K182Q) gene was expressed as a fusion protein with thioredoxin containing a sequence of 6 histidines using the vector pET32d (Invitrogen Sciences) with an intervening sequence representing the protease 3C cleavage site from Escherichia coli. Cultures containing the pET32d-3C-SMEZ-2 M1 gene sequence were grown at 37° C. in terrific broth and induced with the addition of isopropyl thiogalactose to 0.1 mM. Cells were grown to stationary phase, collected by centrifugation, lysed with lysozyme and sonication. Fusion protein was purified by metal chelate chromatography (Ni²⁺ Imminidiacetate sepharose) and eluted with a solution of 100 mM imidazole. The fusion protein was then cleaved with recombinant protease 3C (1 lug/mg or protein) overnight at 4° C. The SMEZ-2 M1 protein was further purified by cation exchange chromatography on carboxymethyl sepharose. Purity was monitored by SDS PAGE. The final product was treated with 10 mM dithiothreitol and dialysed in 20 mM phosphate pH6.0 buffer and stored as aliquots of sterile 5 mg/ml solution at 4° C. until required for coupling.

Coupling of mSAG to Peptides

To couple cysteine containing peptides to the modified SMEZ-2 M1 superantigen, 100 μl 0.2 mM recombinant SMEZ-2 M1 (5 mg/ml) in 20 mM phosphate buffer pH6.0 was incubated with 2 mM C-MOG₃₅₋₅₅ or C-OVA₃₂₃₋₃₉₉ in 0.2 M Tris (pH 8.0), 2 μM CuSO₄ buffer. Conjugation via oxidation and disulphide formation proceeded overnight at RT. Excess uncoupled peptide was removed by 3 repeat washes on a Vivaspin 10K cut-off 0.5 ml concentrator (Vivascience, Lincoln, UK). At each wash, 0.5 ml of phosphate buffered saline was added and the concentrator spun to separate peptide from conjugate. Peptide-coupled products (mSag-MOG₃₅₋₅₅ or mSag-OVA₃₂₃₋₃₃₉) were analysed on 12.5% SDS-Page gels which is capable of separating free SMEZ-2 M1 from conjugate. Typically, no free SMEZ-2 M1 was visible indicating that >99% of the SMZ-2 M1 had been derivatised with the peptide.

To couple peptides to the modified SMEZ-2 M1 superantigen, 0.2 mM recombinant SMEZ-2 M1 was incubated with 2 mM C-MOG₃₅₋₅₅ or C-OVA₃₂₃₋₃₉₉ in 0.2 M Tris (pH 8.0), 2 μM CuSO₄ buffer. Conjugation via oxidation and disulphide formation proceeded overnight at RT. Excess uncoupled peptide was removed using a Vivaspin 0.5 ml concentrator (Vivascience, Lincoln, UK). Peptide-coupled products (mSag-MOG₃₅₋₅₅ or mSag-OVA₃₂₃₋₃₃₉) were analysed on 12% SDS-Page gels and showed that the peptide had bound to mSag, with almost no free peptide or mSag.

Induction and Treatment of EAE

Mice were injected with 20 nmol (50 μg) MOG₃₅₋₅₅ peptide emulsified in CFA (DIFCO Laboratories, Detroit, Mich.) containing 500 μg Mycobacterium tuberculosis (DIFCO Laboratories). The emulsion (100 μl) was injected subcutaneously over the flanks. Mice then received 200 ng pertussis toxin (List Biological, Campbell, Calif.) in pertussis toxin buffer (15 mM Tris, 0.5 M NaCl, 0.017% Triton-X 100) on day 0 and day 2 post-immunization.

Five to seven days after the immunization to induce EAE, or at the peak of the clinical signs of disease (day 18), mice were treated subcutaneously at the back of the neck with 40 pmol (which corresponds to 1 μg) mSag-MOG₃₅₋₅₅ or mSag-OVA₃₂₃₋₃₃₉ emulsified in IFA. In experiments elucidating bystander suppression (FIGS. 6 & 7), mice were immunized and treated as above with 40 pmol of either mSag-MOG₃₅₋₅₅, mSag-MOG₇₉₋₉₆, mSag-PLP₁₀₆₋₁₁₃ or mSag-OVA₃₂₃₋₃₃₉ emulsified in IFA. Immunized mice were observed daily for clinical signs of disease, and scores were assigned as follows: 1, loss of tail tonicity; 2, Limp tail; 3, hind limb weakness; 4, hind limb paralysis; 5, hind limb and forelimb paralysis, moribund state.

Proliferation Assays

Lymph node cells (5×10⁵) isolated from 2D2 or OT-II mice were cultured in 96 well flat bottom plates (BD) for a total of 60 hours with varying concentrations of OVA₃₂₃₋₃₃₉, mSag-OVA₃₂₃₋₃₃₉, MOG₃₅₋₅₅, or mSag-MOG₃₅₋₅₅ (as indicated in FIG. 1B). For the final 12 hours of culture, 0.5 μCi ³[H]-thymidine (Perkin-Elmer Lifesciences Inc, Boston, Mass., USA) was added.

Adoptive Transfer of EAE

Spleen and lymph node cells isolated from mice immunized with MOG₃₅₋₅₅ in CFA and treated with mSag-MOG₃₅₋₅₅ or MOG₃₅₋₅₅ in IFA as described above were isolated 40 days after immunization. Cultures were re-stimulated in vitro using similar conditions as to Ito et al.¹¹ Briefly, cells were seeded at 1×10⁸ in 6 well flat bottom tissue culture plates (Falcon, BD) and cultured for 48 hours with 10 μM MOG₃₅₋₅₅, 50 ng/ml IL-12 (a gift from the Genetics Institute, MA, USA) and 50 ng/ml IL-18 (a gift from the National Institutes of health, Maryland, USA). T cells were then expanded for 48 hours in 100 U/ml IL-2. Blasts (1.3×10⁶/mouse) were injected into naive recipients. The mice were given 200 ng pertussis toxin on the day of immunization and again 48 hours later.

In Vivo Depletion of CD25⁺ Regulatory T Cells

Three days prior to immunization, CD25⁺ cells were depleted (confirmed to be 95% depleted by FACs analysis of blood, spleen and lymph nodes in a pilot experiment up to 10 days after injection) by intraperitoneal injection of 100 μg of the anti-IL2R mAb PC61 antibody (purified from culture supernatants). To control for any non-specific effects of the antibody, control mice were treated with 100 μg of purified normal rat IgG (Sigma Aldrich, Australia)).

Purification of Natural Regulatory CD4⁺/CD25⁺ T Cells

Single cell suspensions of lymph node and/or spleen cells were enriched for cell populations using the AutoMACS system and streptavidin-coated micro-bead separation (Miltenyi Biotec, Bergisch Gladbach, Germany). Cells were incubated with biotinylated anti-CD25 (clone 7D4, PharMingen San Diego, Calif., USA) and further treated according to the manufacturers' protocol for AutoMACS sort. Purified cells were ˜85% CD4⁺/CD25⁺ T cells, as determined by FACs analysis using PE-labelled anti-CD25 (PC61) and PerCP-labelled anti-CD4 (H129.19, both from PharMingen).

In indicated experiments, CD4⁺/CD25⁺ or CD4⁺/CD25⁻ T cells were purified from treated mice. Briefly, C57BL/6J mice were injected with 40 pmol (1 μg) mSag-MOG₃₅₋₅₅ or 40 pmol (1 μg) mSag-OVA₃₂₃₋₃₃₉ (as described above) and 5-7 days later CD4⁺CD25⁺ cells were isolated using the autoMACS procedure outlined above. Then, the indicated cell numbers were transferred intravenously into naive C57BL/6J mice. The mice were then rested for 2-3 days prior to immunization to develop EAE, as described.

Histology

Histology was performed as previously described¹². Briefly, mice were immunized and then treated as described above. Spinal cords were removed towards the end of the experiment (day 35-40 post-immunization) and fixed with 10% buffered formalin (Sigma). Paraffin embedded sections (6 micron thick) were stained with hematoxylin and eosin to assess cellular infiltration.

Statistical Analysis

Results were analysed using the Mann-Whitney U-test for non-parametric data.

Results

A Single Treatment with mSag-MOG₃₅₋₅₅ Suppresses the Development of EAE

The presentation of self-antigen by antigen-presenting cells (APCs) was enhanced by coupling myelin oligodendrocyte glycoprotein (MOG₃₅₋₅₅) self-peptide to a modified version of the streptococcal superantigen SMEZ-2⁶ (mSag) that had been mutated in the TCR binding site to reduce its T cell proliferative activity and had a cysteine introduced to permit disulphide bond coupling. The mSag-MOG₃₅₋₅₅ construct was compared against free MOG₃₅₋₅₅ peptide for its ability to inhibit the generation of encephalitogenic T cells, using the murine experimental autoimmune encephalitis (EAE) model, which is commonly used to study the cause and potential treatments of MS⁷. EAE was induced by immunizing C57BL/6J mice with 20 μmol of MOG₃₅₋₅₅ (50 μg). Mice developed normal symptoms of EAE including disease onset between days 10-15, peak disease around days 20-25 and chronic disease symptoms persisting at day 38 (FIG. 1A). However, one subcutaneous injection of 40 pmol of mSag-MOG₃₅₋₅₅ emulsified in IFA seven days after immunization was sufficient to prevent the onset of disease in the majority of animals. The inhibition of disease was antigen-specific, as treatment with a similar amount of mSag-OVA₃₂₃₋₃₃₉ conjugate; an ovalbumin-derived peptide which also binds to MHC class II IA^{b} had no observable effect on disease severity or onset (FIG. 1A). The mSag-OVA₃₂₃₋₃₃₉ conjugate was not defective because it activated T cells from TCR transgenic mice specific for the OVA₃₂₃₋₃₃₉ peptide more efficiently than the peptide alone (FIG. 1B).

Furthermore, the inhibition of disease was not due to general immuno-modulation by IFA or mSag, as control mice treated with PBS, mSag, or mSag-OVA₃₂₃₋₃₃₉ all emulsified in IFA developed classical EAE (FIG. 1 and data not shown). In addition, free MOG₃₅₋₅₅ peptide could reduce the severity of EAE but required a substantially higher concentration. Whereas 0.04 pmol of mSag-MOG₃₅₋₅₅ inhibited disease in the majority of mice, 0.4 pmol of MOG,₃₅₋₅₅ was ineffective and 4,000 pmol MOG₃₅₋₅₅ was necessary to reduce disease severity (FIG. 1C). Furthermore, in vitro cultured lymph node and spleen cells from mice treated with 4,000 pmol of MOG₃₅₋₅₅ transferred passive EAE, while cells from mice treated with 40 pmol of mSag-MOG₃₅₋₅₅ did not (FIG. 1D). While this confirmed that the suppression of disease was cell mediated, it indicated that the mechanism of suppression mediated by mSag-MOG₃₅₋₅₅ was distinct from that mediated by free MOG₃₅₋₅₅ peptide. A summary of nine different experiments (Table 2) shows that whereas 91% of mice treated with mSag-MOG₃₅₋₅₅ were protected, only 32.7% of untreated mice did not develop EAE (i.e. 67.3% incidence). Taken together, these results show that the modified superantigen presenting the CNS-specific peptide MOG₃₅₋₅₅ specifically prevented the development of EAE (p=0.0005) in a strictly antigen dependent fashion.

Mice treated with mSag-MOG₃₅₋₅₅ were examined for any immune-cell infiltration into the CNS. No inflammatory lesions were observed by histological examination of spinal cord sections from mice treated with mSag-MOG₃₅₋₅₅, but spinal cord sections from mice treated with either mSag-OVA₃₂₃₋₃₃₉, PBS/IFA, or mSag/IFA prior to disease onset all showed evidence of inflammatory lesions in the spinal cord (FIG. 2 and data not shown). These results indicated that treatment with mSag-MOG₃₅₋₅₅ either prevented the development of encephalitogenic T cells altogether, or prevented their entry or accumulation in the CNS.

TABLE 2
Reproducibility of treatment using the mSag-MOG₃₅₋₅₅ complex

| | Treated mice | % Protected | Control mice | % Not sick |
|---|---|---|---|---|
| | (protected/ | of treated | (not sick/ | of control |
| Exp. no | total) | mice | total) | mice |
| 1 | 5/5 | 100 | 0/5 | 0 |
| 2 | 5/5 | 100 | 4/14 | 28.5 |
| 3 | 5/5 | 100 | 2/5 | 40 |
| 4 | 4/8 | 50 | 1/4 | 25 |
| 5 | 5/5 | 100 | 4/10 | 40 |
| 6 | 5/5 | 100 | 5/8 | 63 |
| 7 | 6/8 | 75 | 2/5 | 40 |
| 8 | 9/10 | 90 | 1/5 | 20 |
| 9 | 10/10 | 100 | 3/8 | 37.5 |
| Total | 54/61 | 91%* | 27/96 | 32.7%* |

Summary of experiments performed that involved treatment of mice with the mSag-MOG₃₅₋₅₅ complex and control mice. The percentage of mice which were immunized and then treated with the mSag-MOG₃₅₋₅₅ complex and did not develop EAE has been calculated. In addition, the percentage of control mice that received no treatment and did not develop EAE was calculated to determine the incidence of EAE in house.
*The difference between these two groups is p < 0.0005 (using the Mann-Whitney U test).

mSag-MOG₃₅₋₅₅ Mediated Suppression is Dependent on CD4⁺/CD25⁺ Regulatory T Cells.

The inventors sought to investigate the cellular mechanism of suppression of EAE mediated by mSag-MOG₃₅₋₅₅. Mice were first depleted of CD25⁺ cells by treatment with an anti-CD25 monoclonal antibody, immunized to induce EAE, and then treated with 15 mSag-MOG₃₅₋₅₅ 7 days post-immunization.

Elimination of CD4⁺/CD25⁺ regulatory T cells completely abrogated the suppressive effect of mSag-MOG₃₅₋₅₅. Mice depleted of CD25+ cells then treated with mSag-MOG₃₅₋₅₅ developed comparable disease scores to mice following the standard immunization regime (FIG. 3). The lack of suppression could not be explained by a non-specific effect of the antibody because mice treated with a control rat IgG remained sensitive to mSag-MOG₃₅₋₅₅ (FIG. 3). Thus, mSag-MOG₃₅₋₅₅ suppression required the presence of a population of CD25⁺ cells, most likely, the natural CD4⁺/CD25⁺ regulatory T cells.

Adoptive Transfer of CD4⁺/CD25⁺ Cells from mSag-MOG₃₅₋₅₅ Treated Animals prevents the Onset of EAE

To confirm a direct role for CD25⁺ regulatory T cells in the suppression of EAE mediated by mSag-MOG₃₅₋₅₅, animals were treated with mSag-MOG₃₅₋₅₅ and five days later draining lymph nodes were harvested. CD4⁺/CD25⁺ regulatory T or CD4⁺/CD25⁻ helper T cell populations were isolated using an automated magnetic cell sorting and separation (autoMACS™) system. These cell populations were then adoptively transferred into naive recipients two days prior to immunization with MOG₃₅₋₅₅ in CFA to induce EAE. The experiments showed that animals receiving 2×10⁴ CD4⁺/CD25⁺ cells from mice treated with mSag-MOG₃₅₋₅₅ were completely protected from the development of EAE (FIG. 4). This suppressive effect was limited to the CD25⁺ cell population because mice receiving 2×10⁶ CD4⁺/CD25⁻ cells from mice treated with mSag-MOG₃₅₋₅₅ were not protected.

Furthermore, the data showed clearly that mSag-MOG₃₅₋₅₅ treatment generated antigen-specific CD4⁺/CD25⁺ regulatory T cells in vivo, because mice receiving 10-fold more CD4⁺/CD25⁺ (2×10⁵) regulatory T cells from mSag-OVA₃₂₃₋₃₃₉ treated mice still developed severe EAE (FIG. 4). In addition 2×10⁶ CD4⁺/CD25⁺ regulatory T cells from naïve mice partially suppress the development of EAE (data not shown) confirming previously reported findings⁹. Taken together, these results indicated that the mSag-MOG₃₅₋₅₅ treatment expands and/or activates an existing population of natural regulatory T cells found in naïve mice that are capable of suppressing encephalitogenic T cells in vivo.

Treatment with mSag-MOG₃₅₋₅₅ After the Onset of Disease Symptoms Partially Reverses Established Disease.

To determine whether treatment with the construct of the invention could reverse established disease, EAE was induced in C57BL/6J mice and 18 days later animals were divided into three groups, each group contained mice with an average clinical score of 2. Mice were then treated with a single 40 pmol dose of either mSag-MOG₃₅₋₅₅, mSag-OVA₃₂₃₋₃₃₉, or PBS.

Significant disease reduction was observed in mice treated with mSag-MOG₃₅₋₅₅, but not in the two control groups (FIG. 5). Even though mSag-MOG₃₅₋₅₅ treated mice did not completely recover from disease, a single treatment significantly decreased the clinical scores to a level in which most mice showed a score of 1 or less (loss of tail tonicity).

These results show that the treatment with a single low dose of modified superantigen conjugated to self-peptides effectively activates natural regulatory T cells that completely inhibit the generation of encephalitogenic T cells and moreover reduce existing disease symptoms of EAE in mice. Thus a crippling autoimmune disorder in mice is shown to be completely prevented by the activation of antigen specific Treg cells.

Initial experiments using free peptide to treat disease showed that 10 μg of free MOG₃₅₋₅₅ peptide showed similar degree of disease inhibition as with 1 ng of mSag-MOG₃₅₋₅₅. This result showed that ˜100,000 fold more peptide molecules, compare to mSag-MOG₃₅₋₅₅, were needed to suppress disease (2.7 kDa for MOG35-55 and 27.7 kDa for mSAg-MOG35-55). Furthermore, whereas adoptive transfer of splenocytes from mice treated with mSag-MOG₃₅₋₅₅ protected naive mice from disease upon immunization with MOG₃₅₋₅₅ IN CFA, splenocytes from mice treated with MOG₃₅₋₅₅ did not. This result indicates that mSag-MOG₃₅₋₅₅ inhibits disease with a different mechanism compare to free MOG₃₅₋₅₅ peptide.

Suppression of EAE in F1 (C57BL/6J X B10Q/Ai) Mice

FIG. 1 illustrates that mSAg coupled to MOG₃₅₋₅₅, but not the control peptide OVA₃₂₃₋₃₃₉ can suppress the development of EAE induced with MOG₃₅₋₅₅. This suppression is antigen dependent since the immunizing peptide had to be coupled to mSAg to see any effect. In the autoimmune disease MS and in the disease model EAE there is evidence of both inter and intra molecular epitope spreading as a result of tissue damage. Epitope spreading has important implications for the development of therapies for autoimmune disease.

In this example, the ability of mSAg-peptide to induce bystander suppression to inhibit other encephalitogenic T cells was evaluated in F1 (C57BL/6J X B10Q/Ai) mice.

mSAg Coupled to Peptide can Induce Intramolecular Suppression

To determine whether mSAg coupled to peptide could induce suppression to multiple peptides from the same protein, F1 (C57BL/6J X B10Q/Ai) mice were immunized with MOG₃₅₋₅₅ and then treated seven days later with either mSAg-MOG₃₅₋₅₅ or mSAg-MOG₇₉₋₉₆ emulsified in IFA (FIG. 6). As a negative control one group of animals was treated with mSAg-OVA₃₂₃₋₃₃₉ emulsified in IFA. EAE was prevented in mice treated with both mSAg-MOG₃₅₋₅₅ and mSAg-MOG₇₉₋₉₆, but as expected not with mSAg-OVA₃₂₃₋₃₃₉. This indicates that treatment with mSAg coupled to peptides other than the immunizing peptide is able to induce intramolecular bystander suppression.

mSAg Coupled to Peptide can Induce Intermolecular Suppression

To further address the ability of mSAg coupled to peptide to induce bystander suppression the ability of mSAg-PLP₁₀₃₋₁₁₆ to suppress EAE induced by MOG₃₅₋₅₅ was determined. F1 (C57BL/6J X B10Q/Ai) mice were immunized with MOG₃₅₋₅₅ and treated seven days later with mSAg-MOG₃₅₋₅₅, mSAg-PLP₁₀₃₋₁₁₆ or mSAg alone emulsified in IFA (FIG. 7). Mice treated with mSAg were not protected from the development of EAE, whereas mice treated with mSAg-MOG₃₅₋₅₅ were. Given that PLP₁₀₃₋₁₁₆ was not able to induce EAE in F1 (C57BL/6J X B10Q/Ai) mice, it was hypothesized that this peptide may not play a role in EAE in these mice and hence was not expected to suppress the development of disease. However, mSAg-PLP₁₀₃₋₁₁₆ did suppress the development of EAE to the same extent as mSAg-MOG₃₅₋₅₅. This indicates that treatment with mSAg-peptide can induce intermolecular bystander suppression.

Discussion

The role of regulatory T cells in autoimmune diseases has been investigated extensively over recent years. For example, it has been shown in this study and by others that 2×10⁶ natural regulatory T cells suppress, but do not completely block, EAE upon adoptive transfer into mice prior to immunization with an encephalitogenic peptide⁹. This demonstrates that C57BL/6J mice contain natural myelin-specific CD4⁺/CD25⁺ regulatory T cells capable of suppressing disease. The potential to manipulate these cells as a treatment for autoimmunity has proven difficult due to the inability to induce and/or activate these cells in an antigen specific fashion. The present work reveals that a modified superantigen, with a deleted TCR binding site but intact MHC binding site, coupled to myelin-associated self-peptides not only blocks the development of EAE but can also ameliorate established disease. The mechanism by which treatment with a construct of the invention suppresses disease involves the activation and/or expansion of MOG₃₅₋₅₅-specific natural CD4⁺/CD25⁺ regulatory T cells that are more potent than regulatory T cells from naive mice, as EAE can be suppressed with as few as 2×10⁴ CD4⁺/CD25⁺ T cells after treatment.

The current level of understanding, with regards to the genetic and environmental risk factors for autoimmune diseases, does not allow us to predict susceptibility to disease. Therefore, diseases such as MS are not diagnosed until the patient presents with clinically defined symptoms of disease. Whereas many previous studies have shown an ability to prevent the development of models of autoimmune disease when given prior to the induction of disease, due to the inability to diagnose MS prior to the onset of clinical disease, it is important that potential therapies are effective when the immune response to the auto-antigen is detectable, and/or when overt symptoms of disease are evident. When the treatment with mSag-MOG₃₅₋₅₅ is given after the induction of disease but prior to the onset of clinical symptoms most animals were fully protected from disease development and those that were not, developed mild disease. Treatment after the onset of disease symptoms reduced the severity of disease and prevented disease progression in contrast to controls. While the mSag-MOG₃₅₋₅₅ treatment at this late time point failed to completely cure animals, this result is promising for the development of an antigen specific treatment for autoimmune disease after symptoms are established.

The curative effect of the treatment after the onset of disease may be increased by increasing the dose of mSag-MOG₃₅₋₅₅ administered, using different routes of administration, and/or through the administration of multiple different antigens coupled to mSag as this may allow for the suppression of encephalitogenic T cell responses which may arise due to epitope spreading. Alternatively, combining this therapy with a myelin repair therapy may enhance the success of the treatment at later time points.

Even though the exact number of myelin-specific auto-reactive T cells in patients with MS is not known, most likely they will have several T cell clones each responding to different myelin-derived peptides. Immuno-therapy inhibiting T cells of one specificity only will most likely be inefficient. However, treatment with mSag-MOG₃₅₋₅₅ shows both intra- and inter-molecular myelin suppression (i.e. bystander suppression to different peptides within the same protein and to different peptides from different proteins, respectively). This feature of our invention would overcome the problem that in the majority of MS patients, the specificity of the myelin-reactive T cells is not known.

The invention has been described herein with reference to certain preferred embodiments, in order to enable the reader to practice the invention without undue experimentation. Those skilled in the art will appreciate that the invention is susceptible to variations and modifications other than those specifically described. It is to be understood that the invention includes all such variations and modifications. Furthermore, titles, headings, or the like are provided to enhance the reader's comprehension of this document, and should not be read as limiting the scope of the present invention.

The entire disclosures of all applications, patents and publications, cited above and below, if any, are hereby incorporated by reference.

The reference to any prior art in this specification is not, and should not be taken as, an acknowledgment or any form of suggestion that that prior art forms part of the common general knowledge in the field of endeavour to which the invention relates.

Throughout this specification and any claims which follow, unless the context requires otherwise, the words “comprise”, “comprising” and the like, are to be construed in an inclusive sense as opposed to an exclusive sense, that is to say, in the sense of “including, but not limited to”.

### REFERENCES

1. Hafler, D. A. Multiple sclerosis. J Clin Invest 113, 788-94 (2004)
2. Viglietta, V., Baecher-Allan, C., Weiner, H. L. & Hafler, D. A. Loss of functional suppression by CD4+CD25+ regulatory T cells in patients with multiple sclerosis. J Exp Med 199, 971-9 (2004)
3. Metzler, B. & Wraith, D. Inhibition of experimental autoimmune encephalomyelitis by inhalation but not oral administration of the encephalitogenic peptide: influence of MHC binding affinity. International immunology 5, 1159-1165 (1993)
4. Chen, Y., Kuchroo, V. V., Inobe, J., Hafler, D. & Weiner, H. L. Regulatory T cell clones induced by oral tolerance: Suppression of autoimmune encephalomyelitis. Science 265, 1237-1240 (1994)
5. Apostolou, I. & Von Boehmer, H. In vivo instruction of suppressor commitment in naive T cells. J Exp Med 199, 1401-8 (2004)
6. Proft, T. et al. The streptococcal superantigen SMEZ exhibits wide allelic variation, mosaic structure, and significant antigenic variation. Journal of experimental medicine 191, 1765-1776 (2000)
7. Kuchroo, V. V. et al. T Cell Response in Experimental Autoimmune Encephalomyelitis (EAE): Role of Self and Cross-Reactive Antigens in Shaping, Tuning, and Regulating the Autopathogenic T Cell Repertoire. Annual Review of Immunology 20, 101-123 (2002)
8. Kohm, A. P., Carpentier, P. A. & Miller, S. D. Regulation of experimental autoimmune encephalomyelitis (EAE) by CD4+CD25+ regulatory T cells. Novartis Found Symp 252, 45-52; discussion 52-4, 106-14 (2003)
9. Kohm, A. P., Carpentier, P. A., Anger, H. A. & Miller, S. D. Cutting edge: CD4+CD25+ regulatory T cells suppress antigen-specific autoreactive immune responses and central nervous system inflammation during active experimental autoimmune encephalomyelitis. the Journal of immunology 169, 4712-4716 (2002) 10. Petersen, T. R. et al. A chimeric T cell receptor with super-signaling properties. Int Immunol 16, 889-94 (2004)
11. Proft, T., Moffatt, S. L., Berkahn, C. J. & Fraser, J. D. Identification and Characterisation of Novel Superantigens from Streptococcus pygenes. Journal of Experimental Medicine 189, 89-101 (1999)
12. Nicolson, K. et al. Induction of experimental autoimmune encephalomyelitis in the absence of c-Jun N-terminal kinase 2. Int Immunol 14, 849-56 (2002)
13. McCarron, R. M., R. J. Fallis, and D. E. McFarlin, Alterations in T cell antigen specificity and class II restriction during the course of chronic relapsing experimental allergic encephalomyelitis. J Neuroimmunol, 1990. 29(1-3): p. 73-9
14. Lehmann, P. V., et al., Spreading of T-cell autoimmunity to cryptic determinants of an autoantigen. Nature, 1992. 358(6382): p. 155-7
15. McRae, B. L., et al., Functional evidence for epitope spreading in the relapsing pathology of experimental autoimmune encephalomyelitis. J Exp Med, 1995. 182(1): p. 75-85
16. Klehmet, J., et al., T cell epitope spreading to myelin oligodendrocyte glycoprotein in HLA-DR4 transgenic mice during experimental autoimmune encephalomyelitis. Clin Immunol, 2004. 111(1): p. 53-60
17. Jones, R.E., et al., Epitope spreading is not required for relapses in experimental autoimmune encephalomyelitis. J Immunol, 2003. 170(4): p. 1690-8.
18. Amon, R. and R. Aharoni, Mechanism of action of glatiramer acetate in multiple sclerosis and its potential for the development of new applications. Proc Natl Acad Sci U S A, 2004. 101 Suppl 2: p. 14593-8
19. Miller, A., O. Lider, and H. L. Weiner, Antigen-driven bystander suppression after oral administration of antigens. J Exp Med, 1991. 174(4): p. 791-8.
20. O'Neill E, J., et al., Natural and Induced Regulatory T Cells. Ann N Y Acad Sci, 2004. 1029: p. 180-192
21. Yamazaki S, lyoda T, Tarbell K, Olson K, Velinzon K, Inaba K, Steinman RM. Direct expansion of functional CD25+ CD4+ regulatory T cells by antigen-processing dendritic cells. J Exp Med. 2003 Jul. 21; 198(2):235-47
22. Immunology of Multiple Sclerosis. Sospedra, M. and Martin, R. Annu. Rev. Immunol. 2005. 23:683-747)
23. J Exp Med. 2003 Jul. 21: 198(2): 235-47.
24. J Clin Invest. 2002 Jul.; 110(1):81-90. Multiantigen/multiepitope-directed immune-specific suppression of “complex autoimmune encephalomyelitis” by a novel protein product of a synthetic gene.
25. Zhong M C, Kerlero de Rosbo N, Ben-Nun A
26. Dooley M, Goa K L. Adjuvanted influenza vaccine. Biodrugs 2000 Jul.; 14(1): 61-9

1. A method for the treatment of multiple sclerosis, the method comprising at the least the step of administering to a subject in need thereof an immunomodulatory construct comprising a T cell binding defective superantigen (mSag) coupled to one or more myelin-associated protein, peptide or functionally equivalent variant thereof.

2. A method as claimed in claim 1 wherein the or each myelin-associated protein, peptide or functionally equivalent variant is chosen from the group consisting of:
Myelin oligodendrocyte glycoprotein (MOG);
Myelin-associated glycoprotein (MAG);
Proteolipid protein (PLP);
Myelin basic protein (MBP);
Oligodendrocyte-specific glycoprotein (OSP);
2′,3′-Cyclic nucleotide 3′ phosphodiesterase (CNPase);
Myelin-associated oligodendrocytic basic protein (MOBP);
α-B Crystallin (αB-C);
S100β Protein;
Transaldolase-H (Tal-H);
Oligodendrocyte myelin glycoprotein (OMGP); and
Copaxone®.

3. A method as claimed in claim 2 wherein the or each myelin-associated protein, peptide, or functionally equivalent variant is chosen from the group consisting of:
Myelinoligodendrocyte glycoprotein (MOG);
Proteolipid protein (PLP);
Myelin basic protein (MBP); and
Copaxone®.

4. A method as claimed in claim 1 wherein the mSag is a T cell binding defective version of a bacterial superantigen chosen from the group consisting of:
SMEZ (preferably SMEZ-2);
SPE-C; and
SEA.

5. A method as claimed in claim 4 wherein the mSag is chosen from the group consisting of:
SMEZ-2 W75L;
SMEZ-2 D42C;
SMEZ-2 W75L.D42C.K182Q;
SMEZ-2 Y 18A; and
SMEZ-2 W75L.D42C.K182Q.Y18A.

6. A method as claimed in claim 1 further including administering one or more additional agents to the subject.

7. A method for the treatment of multiple sclerosis, the method comprising at the least the steps of:
a) collecting and purifying regulatory T cells from a patient with multiple sclerosis;
b) ex vivo stimulating the T cells with an immunomodulatory construct comprising a T cell binding defective superantigen (mSag) coupled to at least one second myelin-associated protein, peptide or functionally equivalent variant thereof; and
c) administering the stimulated T cells into the patient.

8. A method of treating multiple sclerosis induced by immune cells to a first myelin-associated protein or peptide, wherein the method includes at least the step of administering to a subject in need thereof an immunomodulatory construct comprising a T cell binding defective superantigen (mSag) coupled to one or more myelin-associated protein, peptide or functionally equivalent variant thereof which is distinct from the first myelin-associated protein or peptide.

9. A method as claimed in claim 1, 7 or 8 wherein the method is for the treatment of relapsing-remitting MS.

10. An immunomodulatory construct comprising a T cell binding defective superantigen coupled to one or more myelin-associated protein, peptide or functionally equivalent variant thereof.

11. A nucleic acid encoding an immunomodulatory construct as claimed in claim 10.

12. A pharmaceutical composition comprising an immunomodulatory construct comprising a T cell binding defective superantigen coupled to one or more myelin-associated protein, peptide or functionally equivalent variant thereof in combination with one or more pharmaceutically acceptable carriers, adjuvants, excipients and/or diluents.

13. A kit for treatment of multiple sclerosis, the kit comprising at least an immunomodulatory construct comprising a T cell binding defective superantigen coupled to one or more myelin-associated protein, peptide or functionally equivalent variant thereof, or a pharmaceutical composition comprising same.
